(19) United States
(12) Patent Application Publication
(10) Pub. No.: US 2003/0003159 A1
(43) Pub. Date: Jan. 2, 2003

(54) Chinese medicinal composition

(76) Inventor: Yongzhong Li, Si Chuan Province (CN)

Correspondence Address: BAKER & BOTTS, 30 ROCKEFELLER PLAZA, NEW YORK, NY, 10112

(21) Appl. No.: 10/151,198

(22) Filed: May 20, 2002

(30) Foreign Application Priority Data
May 25, 2001 (CN) 01108461.8

(51) Int. Cl. A61K 35/78; A61K 35/64; A61K 35/34; A61K 35/37; A61K 35/38

(52) U.S. Cl. 424/538; 424/548; 424/551; 424/735; 424/773; 424/779

(57) ABSTRACT

A Chinese medicinal composition useful in preventing and treating cancer comprises:

(a) 20-30% powder of head and neck of soft-shelled turtle;

(b) 0.05-3% powder of musk;

(c) 5-10% powder of bear gall;

(d) 20-30% powder of achyranthes root;

(e) 0.5-3% powder of bitter almond;

(f) 20-63 % powder of black ant; and

(g) 2-3% powder of rhubarb.

### CROSS-REFERENCE TO RELATED APPLICATIONS

This application claims priority from Chinese Patent Application No. 01108461.8, filed on May 25, 2001 in the Peoples' Republic of China, which is incorporated herein by reference in its entirety.

### BACKGROUND OF INVENTION

1. Field of the Invention

This invention relates to a Chinese medicinal composition, in particular a composition for use in the prevention and treatment of cancer.

2. Background Information

Currently, the treatment of cancer mainly adopts Western medicinal methods, including surgery and radiotherapy. These methods bring great harm to the body but with little effect; in addition, they are simply remedial measures after diagnosis, and cannot prevent oncongenesis of cancer in advance.

The purpose of this invention is to offer a type of Chinese medicine that can prevent and treat cancer, the ingredients of which can be obtained easily and cheaply, which is safe to the body and which has a notable effect in the prevention and treatment of cancer.

### SUMMARY OF THE INVENTION

The Chinese medicine composition of this invention comprises:

(a) 20-30 wt. % of a powder comprising the head and neck of a soft-shelled turtle;

(b) 0.5-3 wt. % of a powder comprising musk;

(c) 5-10 wt. % of a powder comprising bear gall;

(d) 20-30 wt. % of a powder comprising achyranthes root;

(e) 0.5-3 wt. % of a powder comprising bitter almond;

(f) 20-63 wt. % of a powder comprising black ant; and

(g) 2-3 wt. % of a powder comprising rhubarb.

### DETAILED DESCRIPTION OF THE INVENTION

The purpose of the invention is realized as follows. The main ingredients and their weight proportion in the composition of this invention are as follows:

| | |
|---|---|
| Powder of head and neck of soft-shelled | 20-30% |
| turtle | |
| Powder of musk | 0.5-3% |
| Powder of bear gall | 5-10% |
| Powder of achyranthes root | 20-30% |
| Powder of bitter almond | 0.5-3% |
| Powder of black ant | 20-63% |
| Powder of rhubarb | 2-3% |

In the invention, achyranthes root has the effect of nourishing the liver and kidneys, removing stasis and enabling other medicines to be ingested or otherwise absorbed by the body; it can also strengthen bones and muscle physique, and alleviate pains in the heart and abdomen.

Bitter almond in this invention can purge the lung of pathogens, moisten dryness and bring the Qi to lower part of the body, it can prevent the spreading of common colds, bring down the Qi, eliminate phlegm, and cure astriction (i.e. binding) It can also kill helminth and cure tumefaction.

As for musk, it can promote the dispersing function of the lung, thus promoting circulation of main and collateral channels. It can also warm internal organs, detoxify, kill helminth and has a function in abortion.

Bear gall has the function of purging heat. It can cool the heart and liver, improve vision, curing falling sickness and naevus.

Head and neck of soft-shelled turtle can promote the dispersing function of the lung, and promote circulation of main and collateral channels. It can also help other medicines reach the illness and eliminate ulcers, relieve pain and exclude purulence.

Rhubarb can clear away heat and toxic material, promote peristalsis and prevent astriction [?].

This invention can be made into a powder, pill, tablet, aqueous solution or the like. This invention is preferably taken orally.

The composition of this invention is preferably taken three times a day, and each dosage is preferably not less than 15 grams. It can also be used according to the advice of a physician.

Achyranthes root as used in this invention includes cyathula root and common achyranthes root.

The ingredients used in this invention may easily be obtained, have low cost and notable effect.

The invention is further described by the following examples:

### EXAMPLE 1

In a 100 gram composition of this invention, the weight of each ingredient is:

| | |
|---|---|
| Powder of head and neck of soft-shelled | 25 g |
| turtle | |
| Powder of musk | 2 g |
| Powder of bear gall | 8 g |
| Powder of achyranthes root | 30 g |
| Powder of bitter almond | 1 g |
| Powder of black ant | 32 g |
| Powder of rhubarb | 2 g |

### EXAMPLE 2

In a 100 gram composition of this invention, the ingredients and their weight proportion are as follows:

| | |
|---|---|
| Powder of head and neck of soft-shelled | 20 g |
| turtle | |
| Powder of musk | 3 g |
| Powder of bear gall | 5 g |
| Powder of achyranthes root | 20 g |
| Powder of bitter almond | 3 g |
| Powder of black ant | 47 g |
| Powder of rhubarb | 2 g |

In each example, all ingredients are filly mixed with boiling water and concentrated to powder, pill, tablet, aqueous solution, absorbent or the like.

The compositions are taken orally.

The ingredients used in this invention are easily obtained, have a low cost and notable effect.

The invention claimed is:

1. A Chinese medicine which can prevent and treat cancer, wherein the medicine comprises the following ingredients in weight percentage:
(a) 20-30% powder of head and neck of soft-shelled turtle;
(b) 0.05-3% powder of muskiness;
(c) 5-10% powder of bear gall;
(d) 20-30% powder of achyranthes root;
(e) 0.5-3% powder of bitter almond;
(f) 20-63% powder of black ant; and
(g) 2-3% powder of rhubarb.
